SEQUENCE LISTING (67 sequences)

SEQ ID NO: 1 - protein, 16 aa, Homo sapiens
CFFKGADKELLPKMVN

SEQ ID NO: 2 - protein, 16 aa, Homo sapiens
YVCFFKGADKELLPKM

SEQ ID NO: 3 - protein, 14 aa, Homo sapiens
CFFKGADKELLPKM

SEQ ID NO: 4 - protein, 16 aa, Homo sapiens
REPEKQLDNTTENKDF

SEQ ID NO: 5 - protein, 16 aa, Homo sapiens
PEKQLDNTTENKDFGF

SEQ ID NO: 6 - protein, 16 aa, Homo sapiens
KQLDNTTENKDFGFQK

SEQ ID NO: 7 - protein, 16 aa, Homo sapiens
LDNTTENKDFGFQKDK

SEQ ID NO: 8 - protein, 10 aa, Homo sapiens
LDNTTENKDF

SEQ ID NO: 9 - protein, 16 aa, Homo sapiens
KETNINSSENSTSTLS

SEQ ID NO: 10 - protein, 16 aa, Homo sapiens
YRYSKETNINSSENST

SEQ ID NO: 11 - protein, 16 aa, Homo sapiens
YSKETNINSSENSTST

SEQ ID NO: 12 - protein, 16 aa, Homo sapiens
TNINSSENSTSTLSTC

SEQ ID NO: 13 - protein, 16 aa, Homo sapiens
EKDINASENGSVMDEA

SEQ ID NO: 14 - protein, 10 aa, Homo sapiens
TNINSSENST

SEQ ID NO: 15 - protein, 16 aa, Homo sapiens
SIDPMDGNTTENINDT

SEQ ID NO: 16 - protein, 16 aa, Homo sapiens
HRSIDPMDGNTTENIN

SEQ ID NO: 17 - protein, 16 aa, Homo sapiens
DPMDGNTTENINDTFI

SEQ ID NO: 18 - protein, 16 aa, Homo sapiens
MDGNTTENINDTFIKT

SEQ ID NO: 19 - protein, 10 aa, Homo sapiens
MDGNTTENIN

SEQ ID NO: 20 - protein, 15 aa, Homo sapiens
DDQILQLLNETSNSI

SEQ ID NO: 21 - protein, 16 aa, Homo sapiens
QILQLLNETSNSIDNV

SEQ ID NO: 22 - protein, 14 aa, Homo sapiens
QILQLLNETSNSID

SEQ ID NO: 23 - protein, 16 aa, Homo sapiens
NFSEIENQTQNAMDVT

SEQ ID NO: 24 - protein, 16 aa, Homo sapiens
TGNFSEIENQTQNAMD

SEQ ID NO: 25 - protein, 15 aa, Homo sapiens
SEIENQTQNAMDVTT

SEQ ID NO: 26 - protein, 16 aa, Homo sapiens
IENQTQNAMDVTTGYG

SEQ ID NO: 27 - protein, 10 aa, Homo sapiens
IENQTQNAMD

SEQ ID NO: 28 - protein, 16 aa, Homo sapiens
ELSNTSENDEQNAEDL

SEQ ID NO: 29 - protein, 16 aa, Homo sapiens
HCTNISELSNTSENDE

SEQ ID NO: 30 - protein, 16 aa, Homo sapiens
TNISELSNTSENDEQN

SEQ ID NO: 31 - protein, 16 aa, Homo sapiens
ISELSNTSENDEQNAE

SEQ ID NO: 32 - protein, 10 aa, Homo sapiens
ELSNTSENDE

SEQ ID NO: 33 - protein, 16 aa, Homo sapiens
QAINSSENIMDINEEP

SEQ ID NO: 34 - protein, 16 aa, Homo sapiens
KNKEQAINSSENIMDI

SEQ ID NO: 35 - protein, 16 aa, Homo sapiens
KEQAINSSENIMDINE

SEQ ID NO: 36 - protein, 12 aa, Homo sapiens
QAINSSENIMDI

SEQ ID NO: 37 - protein, 16 aa, Homo sapiens
SPKHTPNTSDNEGSDT

SEQ ID NO: 38 - protein, 16 aa, Homo sapiens
EPSPKHTPNTSDNEGS

SEQ ID NO: 39 - protein, 16 aa, Homo sapiens
KHTPNTSDNEGSDTEV

SEQ ID NO: 40 - protein, 16 aa, Homo sapiens
TPNTSDNEGSDTEVCG

SEQ ID NO: 41 - protein, 10 aa, Homo sapiens
TPNTSDNEGS

SEQ ID NO: 42 - protein, 16 aa, Homo sapiens
SLHRYDEGNFFPGSGA

SEQ ID NO: 43 - protein, 16 aa, Homo sapiens
TLEETLTNETRNADDL

SEQ ID NO: 44 - protein, 16 aa, Homo sapiens
EGTLEETLTNETRNAD

SEQ ID NO: 45 - protein, 16 aa, Homo sapiens
EETLTNETRNADDLEV

SEQ ID NO: 46 - protein, 16 aa, Homo sapiens
STKTVMDFVNSSDNVI

SEQ ID NO: 47 - protein, 16 aa, Homo sapiens
KTVMDFVNSSDNVIFV

SEQ ID NO: 48 - protein, 16 aa, Homo sapiens
VMDFVNSSDNVIFVHN

SEQ ID NO: 49 - protein, 12 aa, Homo sapiens
EETLTNETRNAD

SEQ ID NO: 50 - protein, 12 aa, Homo sapiens
VMDFVNSSDNVI

SEQ ID NO: 51 - protein, 16 aa, Homo sapiens
FKSEDSAVNNTQNEDG

SEQ ID NO: 52 - protein, 16 aa, Homo sapiens
SEDSAVNNTQNEDGIA

SEQ ID NO: 53 - protein, 16 aa, Homo sapiens
DSAVNNTQNEDGIAFS

SEQ ID NO: 54 - protein, 12 aa, Homo sapiens
DSAVNNTQNEDG

SEQ ID NO: 55 - protein, 16 aa, Homo sapiens
TDGNETNVTENYELDV

SEQ ID NO: 56 - protein, 16 aa, Homo sapiens
LHTDGNETNVTENYEL

SEQ ID NO: 57 - protein, 16 aa, Homo sapiens
GNETNVTENYELDVAS

SEQ ID NO: 58 - protein, 12 aa, Homo sapiens
GNETNVTENYEL

SEQ ID NO: 59 - protein, 16 aa, Homo sapiens
RDTSINESRNATDESL

SEQ ID NO: 60 - protein, 16 aa, Homo sapiens
EGRDTSINESRNATDE

SEQ ID NO: 61 - protein, 16 aa, Homo sapiens
FATNVTENVLNSSRVQ

SEQ ID NO: 62 - protein, 16 aa, Homo sapiens
RDTSINESRNATDESL

SEQ ID NO: 63 - protein, 16 aa, Homo sapiens
FATNVTENVLNSSRVQ

SEQ ID NO: 64 - protein, 16 aa, Homo sapiens
ILANTSDNAGFCIPEG

SEQ ID NO: 65 - protein, 16 aa, Homo sapiens
YKVPAEILANTSDNAG

SEQ ID NO: 66 - protein, 16 aa, Homo sapiens
VPAEILANTSDNAGFC

SEQ ID NO: 67 - protein, 10 aa, Homo sapiens
ILANTSDNAG

(19) United States
(12) Patent Application Publication
(10) Pub. No.: US 2026/0118356 A1
(43) Pub. Date: Apr. 30, 2026

(54) NOVEL AUTO-ANTIBODIES AND METHOD TO DETECT SJÖGREN'S DISEASE

(71) Applicant: Wisconsin Alumni Research Foundation, Madison, WI (US)

(72) Inventors: Sara MCCOY, Middleton, WI (US); Miriam SHELEF, Madison, WI (US); Michael NEWTON, Madison, WI (US); Zihao ZHENG, Sunnyvale, CA (US)

(73) Assignee: Wisconsin Alumni Research Foundation, Madison, WI (US)

(21) Appl. No.: 19/101,986

(22) PCT Filed: Aug. 9, 2023
(86) PCT No.: PCT/US2023/071892

§ 371 Date: Feb. 7, 2025

Related U.S. Application Data
(60) Provisional application No. 63/396,256, filed on Aug. 9, 2022.

(51) Int. Cl. G01N 33/564

(52) U.S. Cl. CPC G01N 33/564; G01N 2800/101

(57) ABSTRACT

A method and corresponding kit for detecting Sjögren's Disease or predicting labial salivary gland biopsy results. The method includes evaluating an individual for the presence of one or more peptides or whole proteins or fragments thereof having an amino acid sequence selected from SEQ ID NOS: 1-67. The method and kit may be formatted as an enzyme-linked immunosorbent assay (ELISA).

### FEDERAL FUNDING STATEMENT

This invention was made with government support under TR002374 and AR065500 awarded by the National Institutes of Health and under W81XWH-18-1-0717 awarded by the DHA/MRDB. The government has certain rights in the invention.

### SEQUENCE LISTING

The instant application contains a Sequence Listing which has been submitted in an XML file with the USPTO and is hereby incorporated by reference in its entirety. The Sequence Listing was created on Aug. 8, 2023, is named “SEQ_LIST-P220234WO01.xml,” and is 58,788 bytes in size.

### FIELD OF THE INVENTION

Disclosed herein are protein markers that correlate with the presence of Sjögren's Disease in a mammalian subject, including a human subject. Also disclosed herein is a method to diagnose Sjögren's Disease in a mammalian subject by testing the subject for presence of one or more of the protein markers and correlating the presence of the protein marker(s) to Sjögren's Disease in the subject. Also disclosed herein is a kit specifically designed to carry out the method.

### BACKGROUND

Sjögren's Disease is one of the most prevalent systemic rheumatic diseases, affecting an estimated four million Americans. Ninety percent of affected patients are women and, in roughly half of patients, this disorder occurs in the presence of another autoimmune connective tissue disease such as rheumatoid arthritis, lupus, or scleroderma. The defining clinical features of Sjögren's Disease, dryness of the eyes and mouth, arise from an autoimmune process affecting the lacrimal and salivary glands. Sjögren's Disease can cause significant dysfunction in a variety of organs/systems and is associated with significant morbidity and increased risk of lymphoma. There is no FDA-approved disease modifying therapy. An antibody-focused test (for anti-Ro (SS-A)) is available that, in combination with other clinical indicators, can be used to diagnose Sjögren's Disease. However, 30% of Sjögren's patients are “seronegative” and require an invasive inner lip biopsy to look for signs of inflammation within the exocrine glands (salivary and lacrimal) in order to make the diagnosis. To help replace the need for an invasive lip biopsy, the present disclosure develops a new diagnostic assay for Sjögren's Disease based on discovery of novel autoantibodies relevant to the disease process.

### SUMMARY

Sjögren's Disease (“Sjögren's” or “SjD”) is typically diagnosed by the presence of an anti-SSA antibody (“SSA+”) or focal lymphocytic sialadenitis in salivary gland tissue. Among Sjögren's patients who are anti-SSA antibody negative (“SSA-”), a salivary gland biopsy with lymphocytic infiltrate is required for diagnosis. Disclosed herein are novel autoantibodies that positively correlate with the presence of Sjögren's in SSA− subjects. Thus, disclosed herein is a method of diagnosing Sjögren's Disease in a mammalian subject by testing the subject for the presence and/or concentration of one or more of these newly discovered antibodies. Currently SSA− patients can be diagnosed only via a painful and intrusive salivary gland biopsy. Furthermore, practitioners capable of performing the biopsy and specialists able to interpret results are not widely available. Thus, disclosed herein is a non-invasive, readily available means to diagnose Sjögren's Disease, especially in SSA− subjects.

Thus, disclosed herein is a method for detecting Sjögren's Disease or predicting labial salivary gland biopsy results, the method comprising:
a) providing a liquid sample obtained from an individual;
b) contacting the sample with a peptide or whole protein or fragment thereof that comprises an amino acid sequence selected from the group consisting of SEQ ID NOS: 1-67 or an epitope from the peptide, whole protein, or fragment thereof containing these sequences, under conditions appropriate to form a complex between at least a portion of any antibodies in the sample that are specific for one or more of the peptide, whole protein, or fragment thereof, and
c) correlating an amount of the complex formed in step b) to detection of Sjögren's Disease or predicting labial salivary gland biopsy results in the individual.

The liquid sample is whole blood, blood plasma, or blood serum. The sample is contacted with the protein in an enzyme-linked immunosorbent assay format.

Also disclosed herein is a kit comprising, in combination, at least one peptide or whole protein or fragment thereof comprising an amino acid sequence selected from the group consisting of SEQ ID NOS: 1-67 adhered to a support, reagents suitable for performing an enzyme-linked immunosorbent assay, and directions for use of the kit.

### BRIEF DESCRIPTION OF THE DRAWINGS

FIG. 1. Consort flow diagram demonstrating peptide selection for external validation.

FIGS. 2A-2O. Internal validation of select peptides identified from the whole peptidome array. ELISA for each peptide was performed using sera from SSA− Sjögren's (n=8) and control (n=8) subjects. FIG. 2A=DTD2. FIG. 2B=RESF1. FIG. 2C=SCRB2. FIG. 2D=BRWD1. FIG. 2E=PDZD8. FIG. 2F=SLK. FIG. 2G=GPAT1. FIG. 2H=SO1B1. FIG. 2I=CYP7A1. FIG. 2J=LRBA. FIG. 2K=HDAC9. FIG. 2L=NPAT. FIG. 2M=TEX15. FIG. 2N=LRCC1. FIG. 2O=KNL1.

FIGS. 3A-3C. Novel IgG binding to peptides by ELISA among SSA− Sjögren's, autoimmune- and sicca-controls (n=45 SSA− Sjögren's subjects, n=41 sicca controls, and n=41 autoimmune controls). FIG. 3A shows that SSA− Sjögren's subjects bind peptides from DTD2 and RESF1 more than SICCA controls. FIG. 3B shows that SSA− Sjögren's subjects bind peptides from DTD2 and RESF1 more than combined sicca and autoimmune controls. Recognizing the directional changes from the array data, FIG. 3C shows a one-sided Wilcoxon rank-sum test with Benjamini-Hochberg correction (q-value) to control the false discovery rate.

FIGS. 3D-3E. Novel IgG binding to peptides by ELISA from focus score positive salivary gland biopsy compared to focus score negative salivary gland biopsies. N=85 focus score positive and N=107 focus score negative salivary gland biopsies. FIG. 3D shows area under the ROC curve (AUC) comparing distributions of adjusted optical density of peptide groups comparing between FS positive vs. negative biopsies (n=85 FS positive and n=107 FS negative). The forest plot shows the degree of IgG binding to the peptide of interest differed between focus score positive and focus score negative comparisons. FIG. 3E shows a one-sided Wilcoxon rank-sum test with Benjamini-Hochberg correction (q-value) to control the false discovery rate.

FIG. 4A shows the logistic regression model that has AUC of 73.5% (95% CI: 66.0-79.9%), which decreased to 72.2% after adjusting for optimism. FIG. 4B has the dot plot showing the separation between SSA− SjD and combined controls by SjD prediction model score. FIG. 4C shows that on cross validation, the models using clinical predictors plus IgG binding to DTD2 had better overall prediction accuracy than models that used only clinical variables. FIGS. 4D-4E show specificity and sensitivity graphed separately for cut-points of the score ranging from −1.6 to 1.6. Optimism-corrected values are shown as dotted lines and differ from original values by at most 2.6 or 1.8% for sensitivity and specificity, respectively; FIGS. 4F-4G show positive and negative predictive value graphed separately.

FIG. 5A shows the final model incorporated four predictors (binding a peptide form DTD2, unstimulated salivary flow, platelet count, and high ANA) with an AUC of 71.6% (95% CI: 63.9-78.2%). The table shows estimated model coefficients and their standard errors in subscript. The effects of single term deletion are shown. FIG. 5B is a dot plot showing the separation between positive and negative focus score groups. FIG. 5C shows that on cross validation, the models using clinical predictors plus IgG binding to DTD2 had better overall prediction accuracy than models that used only clinical variables. FIGS. 5D-5E show specificity and sensitivity graphed separately for cut-points of the score ranging from −1.6 to 1.6. Optimism-corrected values are shown as dotted lines and differ from original values by at most 2.6 or 1.8% for sensitivity and specificity, respectively. FIGS. 5F-5G show positive and negative predictive value graphed separately, and novel IgG binding to peptides by ELISA among SSA− Sjögren's, autoimmune- and sicca-controls (n=45 SSA− Sjögren's subjects, n=41 sicca controls, and n=24 autoimmune controls).

### DETAILED DESCRIPTION

The disclosure is based on the identification of autoantibodies that correlate with the presence of Sjögren's Disease in a mammalian subject, including a human subject. Using whole peptidome array technology, 15 peptides were identified that can be found in patient serum and used to diagnose patients who are SSA− (Table 1). Thus, disclosed herein are protein markers that correlate with the presence of Sjögren's Disease in a mammalian subject, including a human subject. Also disclosed herein is a method to diagnose Sjögren's Disease in a mammalian subject by testing the subject for presence of one or more of the protein markers and correlating the presence of the protein marker(s) to Sjögren's Disease in the subject. Also disclosed herein is a kit specifically designed to carry out the method.

On aspect of the method comprises contacting a sample obtained from an individual with a peptide or whole protein or fragment thereof that comprises an amino acid sequence selected from the group consisting of SEQ ID NOS: 1-67 (Table 1) or an epitope from the peptide, whole protein, or fragment thereof containing these sequences, under conditions appropriate to form a complex between at least a portion of any antibodies in the sample that are specific for one or more of the peptide, whole protein, or fragment thereof.

In the method provided herein, the test sample can be in liquid phase, such as whole blood, blood plasma, blood serum, saliva, tears, or other bodily fluid. The sample can be diluted or concentrated or subjected to one or more processing steps. The detection can be by an immunological assay, described in further detail below, such as ELISA performed in any of a wide variety of formats.

### Definitions

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the method and kits disclosed herein shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and kits disclosed herein can comprise, consist of, or consist essentially of the essential elements and limitations of the method described herein, as well as any additional or optional ingredients, components, or limitations described herein or otherwise useful in immunology and detecting antibodies and other proteins specifically. The method disclosed herein may be practiced in the absence of any element or step which is not specifically disclosed herein.

“Antibody” refers to a polypeptide ligand substantially encoded by an immunoglobulin gene or immunoglobulin genes, or fragments thereof, which specifically recognizes and binds a molecule or a region or domain of a molecule (an epitope). The recognized imnmunoglobulin genes include the kappa and lambda light chain constant region genes, the alpha, gamma, delta, epsilon and mu heavy chain constant region genes, and the myriad inimunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies.

An “autoantibody” is an antibody present in an individual that specifically recognizes a biomolecule present in the individual. Typically, an autoantibody specifically binds a protein expressed by the individual, or a modified form thereof present in a sample from the individual. Autoantibodies are generally IgG antibodies that circulate in the blood of an individual, although the disclosure is not limited to IgG autoantibodies or to autoantibodies present in the blood.

The term “epitope” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996). Two antibodies are said to bind to the same epitope of a protein if amino acid mutations in the protein that reduce or eliminate binding of one antibody also reduce or eliminate binding of the other antibody, and/or if the antibodies compete for binding to the protein, i.e., binding of one antibody to the protein reduces or eliminates binding of the other antibody.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “antigen” as used herein refers to proteins or polypeptides to be used as targets for screening test samples obtained from subjects for the presence of antibodies. The antigen is contemplated to include any fragments thereof of the so-identified proteins, in particular, immunologically detectable fragments. The term antigen is also meant to include immunologically detectable products of proteolysis of the proteins, as well as processed forms, post-translationally modified forms, as well as sequence variants, including but not limited to allelic variants and splice variants of the antigen or fragments thereof. The identification or listing of antigens also includes amino acid sequence variants of these, for example, sequence variants that include a fragment, domain, or epitope that shares immune reactivity with the identified antigen. The fragment, domain, or epitope can be provided as part of or attached to a larger molecule or compound.

A “variant” of a polypeptide or protein, as used herein, refers to an amino acid sequence that is altered with respect to the referenced polypeptide or protein by one or more amino acids. In the present disclosure, a variant of a polypeptide retains the antibody binding property of the referenced protein. In preferred aspects of the disclosure, a variant of a polypeptide or protein can be specifically bound by the same population of autoantibodies that are able to bind the referenced protein. Preferably a variant of a polypeptide has at least 60% identity to the referenced protein over a sequence of at least 10 amino acids. More preferably a variant of a polypeptide is at least 70% identical to the referenced protein over a sequence of at least 4 amino acids. Protein variants can be, for example, at least 80%, at least 90%, at least 95%, at least 96%, at least 97%, at least 98%, or at least 99% identical to referenced polypeptide over a sequence of at least 4 amino acids. Protein variants of the disclosure can be, for example, at least 80%, at least 90%, at least 95%, at least 96%, at least 97%, at least 98%, or at least 99% identical to referenced polypeptide over a sequence of at least 10 amino acids. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may also include amino acid deletions or insertions, or both. Guidance in determining which amino acid residues may be substituted, inserted, or deleted without abolishing immunological reactivity may be found using computer programs well-known in the art, for example, DNASTAR software.

The phrase “specifically (or selectively) binds” to an antibody when referring to a protein or peptide, refers to a binding reaction that is determinative of the presence of the protein in a heterogeneous population of proteins and other biologics. Thus, under designated immunoassay conditions, the specified antibodies bind to a particular protein at a level that is statistically significantly different from background, and do not substantially bind in a significant amount to other proteins present in the sample.

“Sensitivity” is defined as the percent of diseased individuals in which the biomarker of interest is detected. Nondiseased individuals diagnosed by the test as diseased are “false positives.”

“Specificity” is defined as the percent of nondiseased individuals for which the biomarker of interest is not detected. Diseased individuals not detected by the assay are “false negatives.” Subjects who are not diseased and who test negative in the assay, are termed “true negatives.”

### METHODS

### Population

We used sera from the same subjects on a whole peptidome array for ELISA internal validation. The array included serum from eight (8) SSA-positive (“SSA+”) and eight (8) SSA-negative (“SSA−”) SjD patients meeting SjD ACR/EULAR criteria (IRB #2021-0945 and #2015-0156). Shiboski S C, Shiboski C H, Criswell L, et al. American College of Rheumatology classification criteria for Sjögren's syndrome: a data-driven, expert consensus approach in the Sjögren's International Collaborative Clinical Alliance cohort. Arthritis Care Res. (Hoboken) (2012) 64(4):475-87. (See Table 2 below). All the SjD array subjects were white females. For external validation of the array, we used sera from subjects that were not included on the whole peptidome array.

For external validation, we used samples from the SICCA registry cohort (IRB #2021-0945). The SICCA registry, a National Institutes of Health-funded registry, is a multisite international registry housed at the University of California, San Francisco. Participants were referred to the registry if:
i) they had a known diagnosis of SjD;
ii) salivary gland enlargement;
iii) repeated dental caries without risk factors; or
iv) abnormal serology (anti-SSA or anti-SSB antibody, antinuclear antibody [ANA], or rheumatoid factor [RF]).

Further registry details can be found at siccaonline.ucsf.edu or as described in prior publications. See Shiboski S C, Shiboski C H, Criswell L, et al. American College of Rheumatology classification criteria for Sjögren's syndrome: a data-driven, expert consensus approach in the Sjögren's International Collaborative Clinical Alliance cohort. Arthritis Care Res. (Hoboken) (2012) 64(4):475-87. Daniels T E, Criswell L A, Shiboski C, et al. An early view of the international Sjögren's syndrome registry. Arthritis Rheum. (2009) 61(5):711-4. McCoy S S, Sampene E, Baer A N. Association of Sjögren's Syndrome With Reduced Lifetime Sex Hormone Exposure: A Case-Control Study. Arthritis Care Res. (Hoboken) (2020) 72(9):1315-22.

SSA− SjD subjects met ACR/EULAR criteria. We compared SSA− SjD subjects to Sicca-controls and autoimmune-controls. Sicca-controls had symptoms or signs of dryness but lacked autoimmunity (ANA<1:320, negative RF, negative anti-SSA antibodies, and focus score<1 on labial salivary gland biopsy). Autoimmune controls had autoimmune features (ANA≥1:320, positive RF, or focus score≥1 on labial salivary gland biopsy) but did not meet the 2016 ACR/EULAR criteria for SjD.

### Whole Peptidome Array and Analysis

To broadly evaluate autoantibody reactivity to identify common features of antigens and better understand SjD, we used a whole peptidome array and contracted with Roche NimbleGen (Madison, WI, USA) for the performance of and the data resulting from the array. The array was generated by covalently anchoring peptides to a chip at their C-terminals. Amino acids were added sequentially by C-to-N terminal chain extension. Sixteen-(16)-amino acid peptides were tiled every two amino acids across the whole human peptidome. Because the position of each peptide on the array chip was known, the location and intensity of the binding signal on the chip can be recorded and a peptide response pattern for the target protein could be constructed. The whole peptidome array was previously validated using RA samples. See Zheng Z, Mergaert A M, Fahmy L M, et al. Disordered Antigens and Epitope Overlap Between Anti-Citrullinated Protein Antibodies and Rheumatoid Factor in Rheumatoid Arthritis. Arthritis Rheumatol (2020) 72(2):262-72. The peptidome array comprises over 5.3 million peptides by overlapping 16 amino acids tiled at 2 amino acid intervals across the human proteome. In addition to the 16 SjD subjects, the array included sixteen (16) systemic lupus erythematosus (“SLE”) subjects meeting the 2012 Systemic Lupus International Collaborating Clinics (SLICC) criteria and eight (8) subjects meeting 2010 ACR/EULAR criteria for rheumatoid arthritis (“RA”). For the SLICC criteria, see Petri M, Orbai A M, Alarcón G S, et al. Derivation and validation of the Systemic Lupus International Collaborating Clinics classification criteria for systemic lupus erythematosus. Arthritis Rheum. (2012) 64(8):2677-86. For the ACR/EULAR criteria, see Aletaha D, Neogi T, Silman A J, et al. 2010 Rheumatoid Arthritis Classification Criteria: an American College of Rheumatology/European League Against Rheumatism Collaborative Initiative. Ann. Rheum. Dis. (2010) 69(9):1580-8. Each autoimmune disease subject had an age- and sex-matched control with some control subjects serving as a control for more than one autoimmune disease subject. We developed novel statistical methodologies to optimally analyze this large data set. Although methods to analyze large data sets on gene expression already exist, antibody binding to peptide arrays have different sampling features and require different techniques to differentiate signal from noise. To better account for the variance uncertainties across the peptide array, we used a large-scale testing tool, MixTwice, to compare the mean difference in signal intensity between two groups. See Zheng Z, Mergaert A M, Ong I M, Shelef M A, Newton M A. MixTwice: large-scale hypothesis testing for peptide arrays by variance mixing. Bioinformatics (2021) 37(17):2637-43. The tool is available online at cran.r-project.org/web/packages/MixTwice/index.html. Provided with the estimated effect size and the estimated standard error of a two-sample t test, MixTwice advances an empirical Bayes tool to calculate the local false discovery rate (locFDR), the probability of null given the data vector using non-parametric maximum likelihood estimation (MILE) with shape constraint. Also, we used the r-value methodology as a ranking statistic for the effect size of a peptide over the whole array.

We assigned peptides a local false discovery rate (locFDR) for sensitive filtering, combined with data on binding affinity, protein context, and peptide sequence. We defined a nearest neighbor (NN) peptide as on the same protein and at the immediate neighboring position. The nearest-neighbor locFDR (NN-locFDR) of a certain peptide is the averaged locFDR of its NN peptide(s). We used a combination of r-value<0.01, locFDR<0.01, and nearest neighbor locFDR<0.05 on peptides transformed by an empirical cumulative distribution function and found 387 seropositive and 469 seronegative peptides bound more than controls. Within the array, two peptides were identified in the comparison of SSA+vs. combined control and one in SSA− vs. combined control.

### Selection of Top Candidate Peptides

We prioritized peptides for individual analysis by selecting those peptides with at least two significant peptides bound in a protein and with a fold change of 10. After removing peptides with fold change expression<10 and peptides with <two of the same proteins bound, we focused our analysis on peptides where most ≥50% SjD subjects showed significant increase over control subjects.

### Enzyme-Linked Immunosorbent Assay (“ELISA”)

With knowledge of the relevant targets, an ELISA can be created using any suitable format. ELISA's generally utilize antigen-specific monoclonal antibodies in concert with a specific antibody-enzyme conjugate to detect a protein target and (optionally) to quantify the concentration of the protein target. ELISA's may be run in a qualitative or quantitative format. Qualitative results provide a simple positive or negative result (yes or no) for a sample. The cutoff between positive and negative is determined empirically, to maximize sensitivity, specificity, or both.

The basic “direct” ELISA format itself is conventional and known since the 1970's. See Engvall, E. (1972-11-22). “Enzyme-linked immunosorbent assay, Elisa,” The Journal of Immunology 109 (1):129-135. The general protocols will not be discussed in any detail herein. For a full treatment, see, for example, “Enzyme-Linked Immunosorbent Assay (ELISA): From A to Z” (SpringerBriefs in Applied Sciences and Technology), Amit Kumar and Allam Appa Rao, Eds., © 2018, Springer (Singapore), ISBN 978-9811067655. The direct ELISA protocol has been modified over the years to yield different types of ELISA's, any of which can be used to detect and quantify the protein targets disclosed herein. These additional ELISA formats include indirect, antibody sandwich, double antibody-sandwich, and competitive ELISA's.

The fundamental protocol for our indirect ELISA is as follows: After selection of the candidate peptides sequences, we submitted the sequences for synthesis of biotinylated peptides through a commercial supplier, Biomatik Corporation (Kitchener, Ontario, Canada). Peptides were dissolved per specifications provided by Biomatik to a concentration of 500 ng/mL. ELISA plates were coated with streptavidin and incubated overnight at 4° C. The next day, the plate was washed twice with PBS. Next, the biotinylated peptide was then added to the plate in a 1:500 dilution and incubated at room temperature for 1 hour. After 1 hour, the plate was washed three times with 0.2% Tween-20 in PBS. The wells were then blocked with 5% non-fat dried milk in 0.2% Tween-20 in PBS for 2.5 hours at room temperature. Next, the serum samples and plate controls were diluted 1:100 in 5% non-fat dried milk in 0.2% Tween-20 in PBS and added in duplicate to the plate overnight at 4° C. The following day, the plate was washed 4 times with 0.2% Tween-20 in PBS. The HRP-conjugated mouse anti-human IgG clone JDC-10 was diluted 1:5000 in 5% non-fat dried milk in 0.2% Tween-20 in PBS for 1 hour at room temperature in the dark. Next, the plate was washed 4 times with 0.2% Tween-20 in PBS. Finally, TMB-Slow ELISA formulation (ThermoFisher Scientific, Coraopolis, Pennsylvania, USA, catalog no. 34024) was added to each well and developed in the dark at room temperature for 15 minutes. 0.2 M H₂SO₄ stop solution was then added to stop the reaction and the plate was read at 450 and 540 nm. For analysis, we subtracted a no peptide with serum control (accounting for non-specific background plate binding), a peptide with no serum control (to account for absorbance from the peptides), and we normalized between plates using a positive control.

### Statistical Analysis

We used the Mann-Whitney-Wilcoxon rank-sum test for hypothesis testing of the ELISA data on given the nonparametric nature of our data. (Mann, Henry B.; Whitney, Donald R. (1947) “On a Test of Whether one of Two Random Variables is Stochastically Larger than the Other,” Annals of Mathematical Statistics 18(1):50-60.)

### Results

### Whole Peptidome Array Analysis

Of more than 5.3 million peptides, our analysis using our whole human peptidome analysis yielded 469 differentially bound peptides comparing SSA− to control subjects. Of these, 299 were excluded because they had a fold change <10; 152 were excluded because they lacked significant binding to at least two peptides in the same protein, and six failed internal validation. Our final validation analysis included fifteen (15) total peptides (Table 1), selected from 30 total significant peptide sequences that were identified as significant on the array.

### Internal Validation

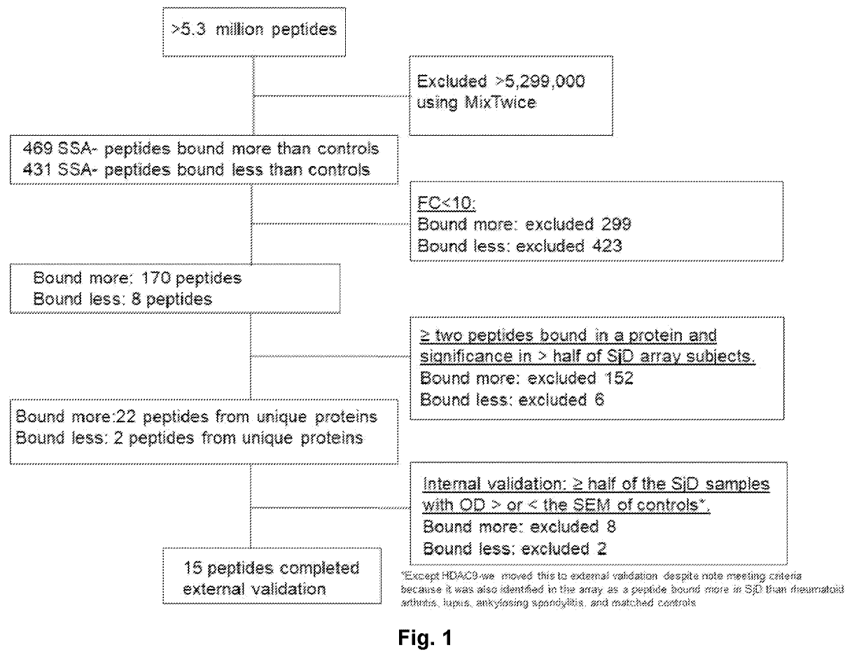

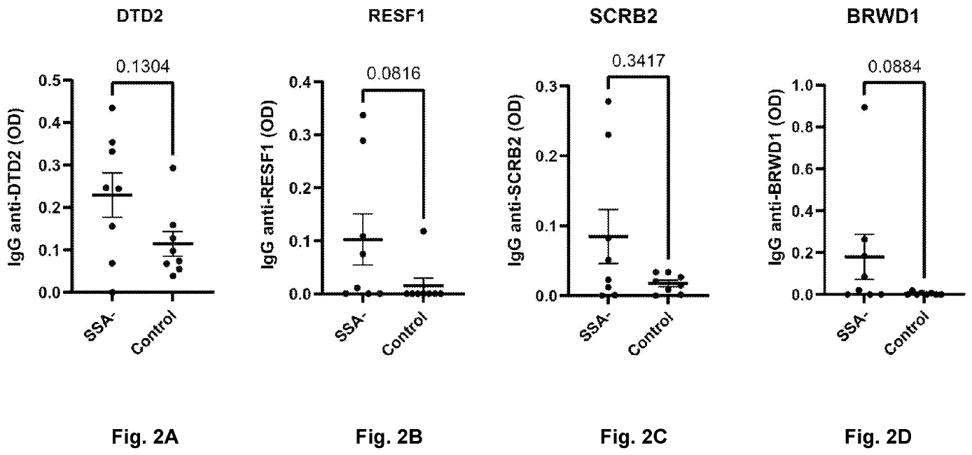

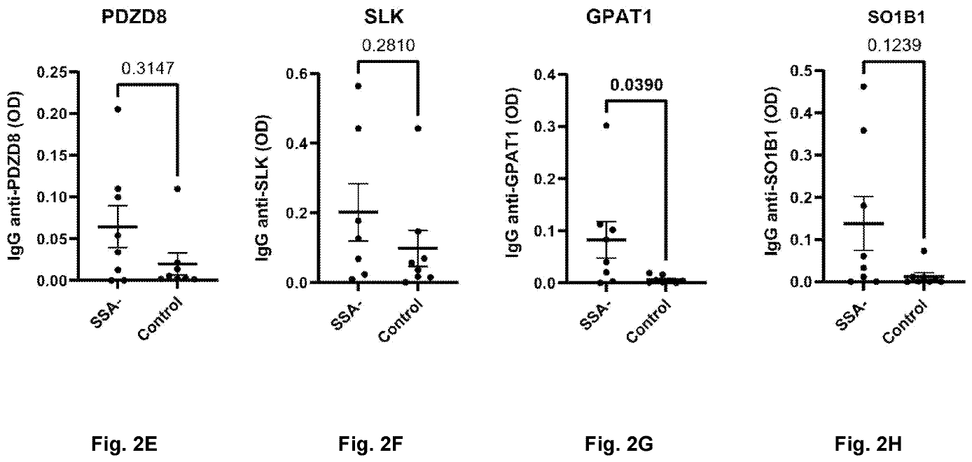

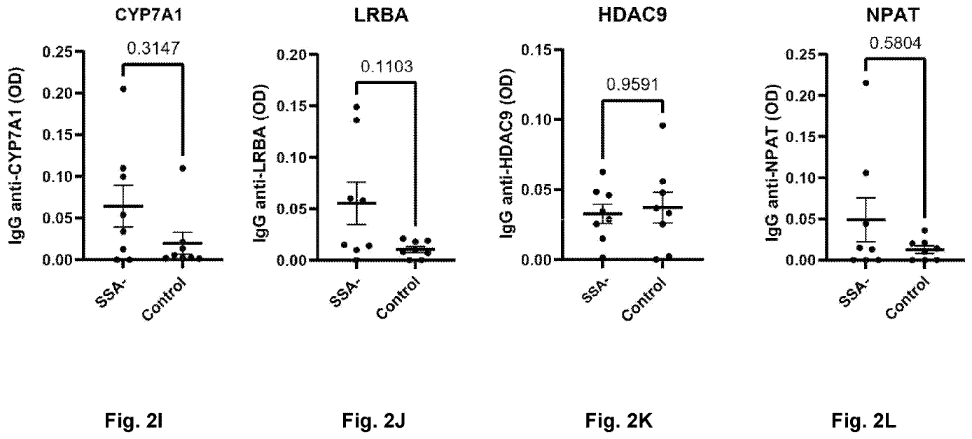

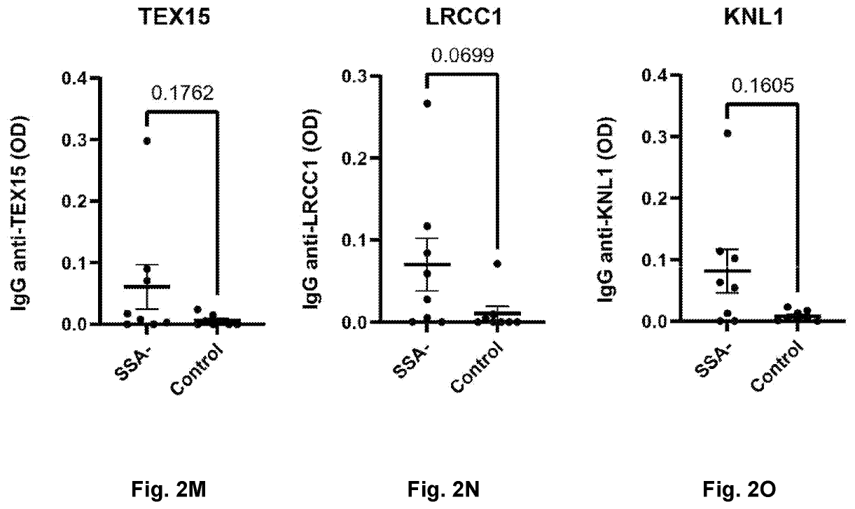

Using sera from SSA− Sjögren's patients (n=8; Table 2) and age- and sex-matched controls (n=8), IgG binding to a high density whole human peptidome array was quantified. The highest bound peptides from the array, as defined by our whole human peptidome analysis method, were internally validated by ELISA using sera from the same subjects. See FIG. 1 and FIGS. 2A-2H. (Zihao Zheng, Aisha M. Mergaert, Irene M. Ong, Miriam A. Shelef, Michael A. Newton (1 Sep. 2021) “MixTwice: large-scale hypothesis testing for peptide arrays by variance mixing,” Bioinformatics, 37(17): 2637-2643; doi.org/10.1093/bioinformatics/btab162.)

### External Validation and Test Performance

Based on results from internal validation, fifteen (15) peptides were selected for ELISA external validation using sera from the following age-, sex-, and race-matched groups from the Sjögren's International Collaborative Clinical Alliance (“SICCA”) biorepository (siccaonline.ucsf.edu/home) (Table 2): (1) SSA− Sjögren's subjects (met the 2016 American College of Rheumatology/European Alliance of Associations for Rheumatology (“ACR/EULAR”) criteria for Sjögrens; n=76), (2) SICCA controls (SICCA with negative anti-nuclear antibody test (“ANA”), negative rheumatoid factor, negative SSA, and focus score<1; n=75), and (3) autoimmune controls (positive ANA (≥1:320), rheumatoid factor positive, or SSA positive, but fail to meet the 2016 ACR/EULAR criteria for Sjögren's; n=38). ELISA results were compared using nonparametric testing with Mann-Whitney-Wilcoxin rank-sum test. We performed adaptive shrinkage with Lasso regression to select peptides for a random forest model to predict SSA− Sjögren's in the test subjects.

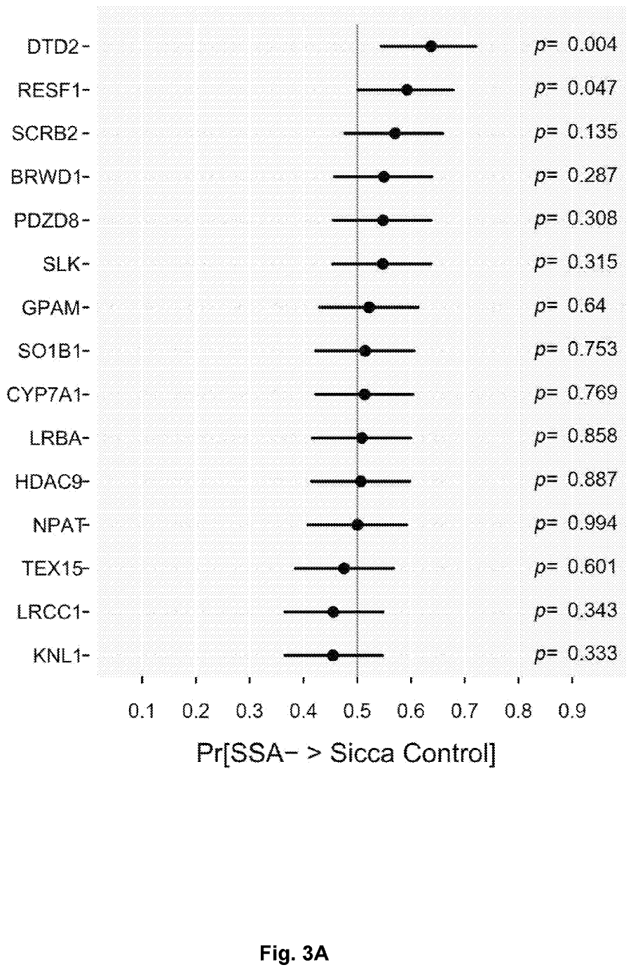

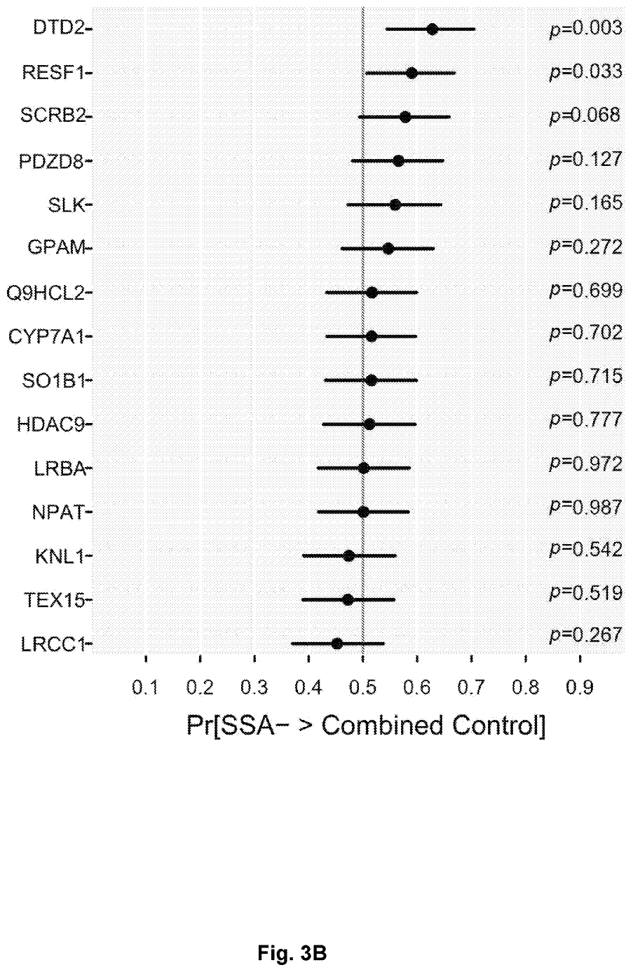

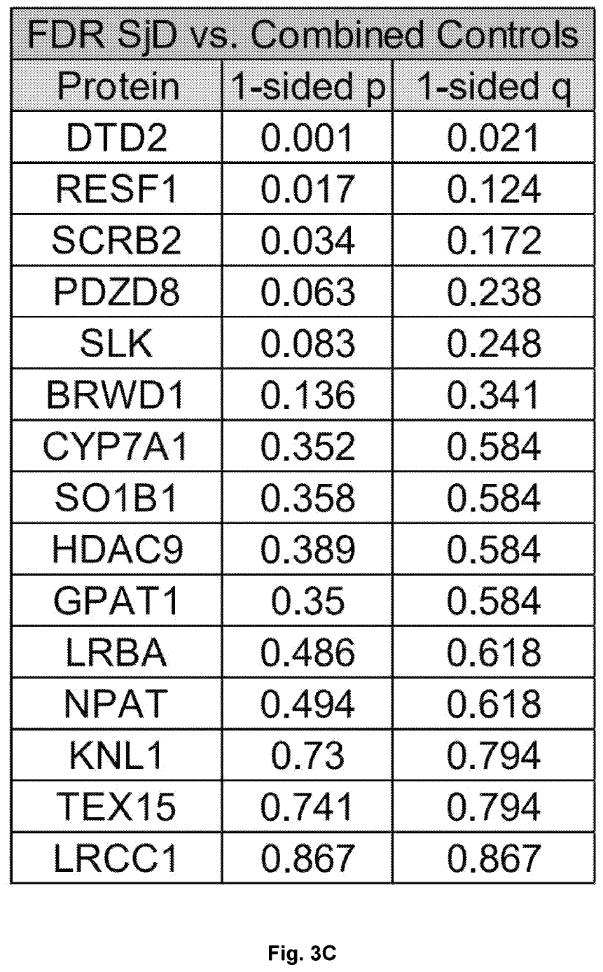

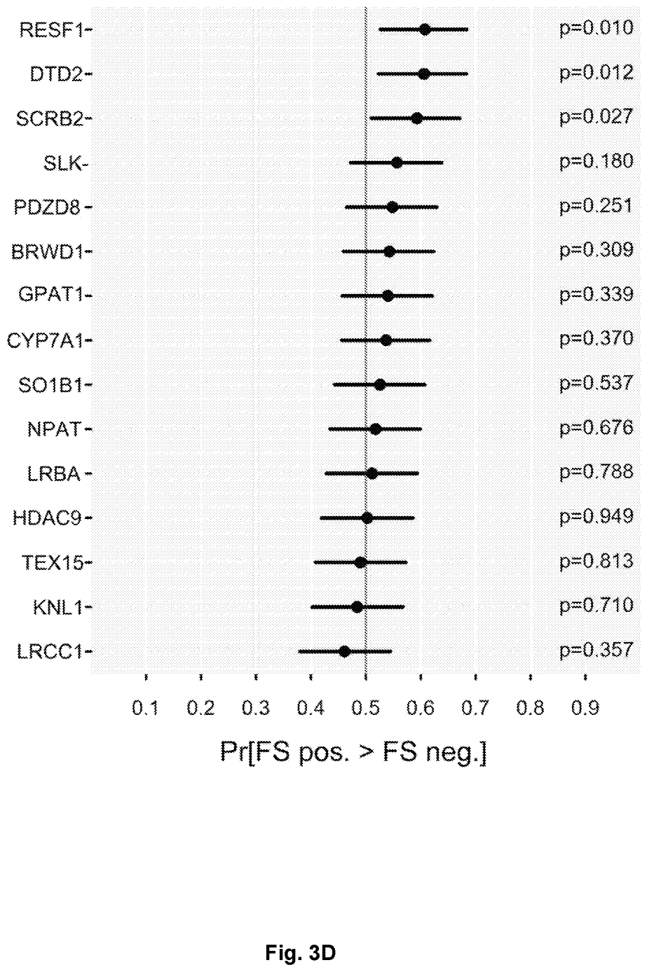

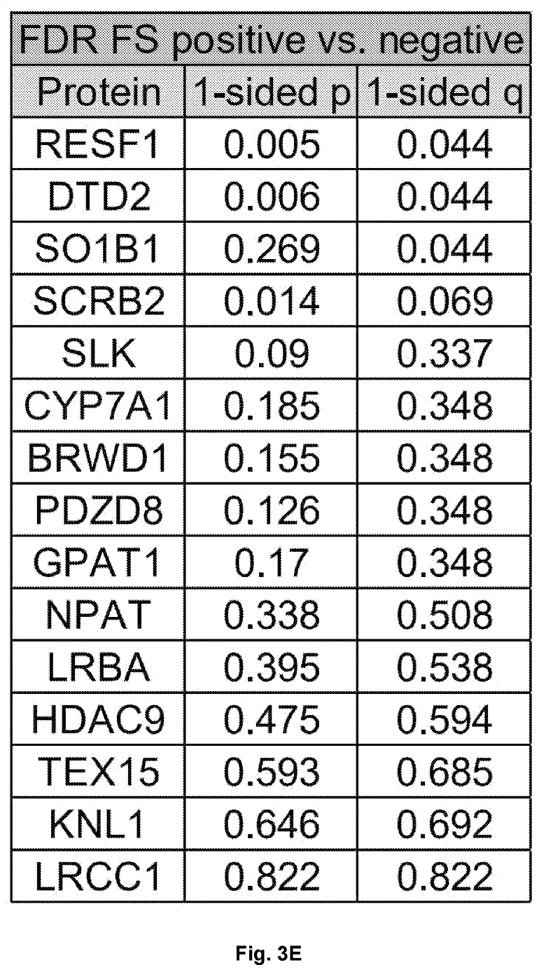

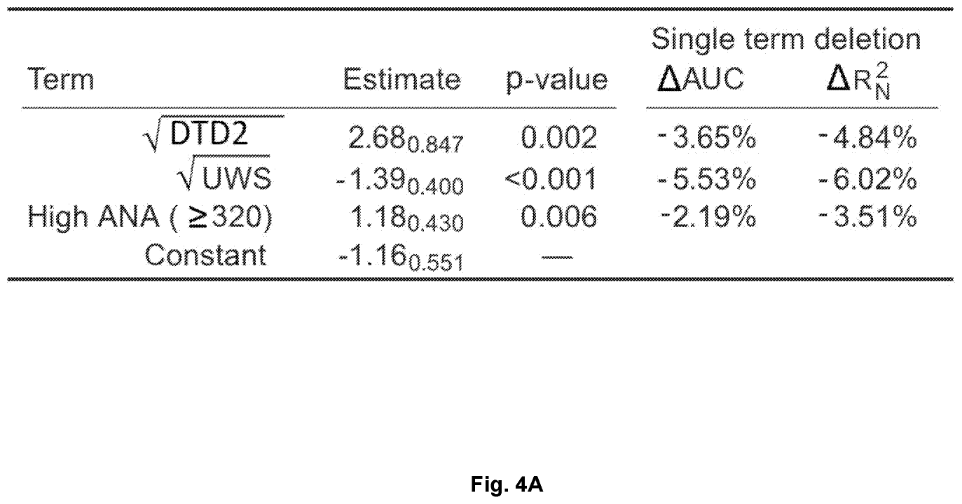

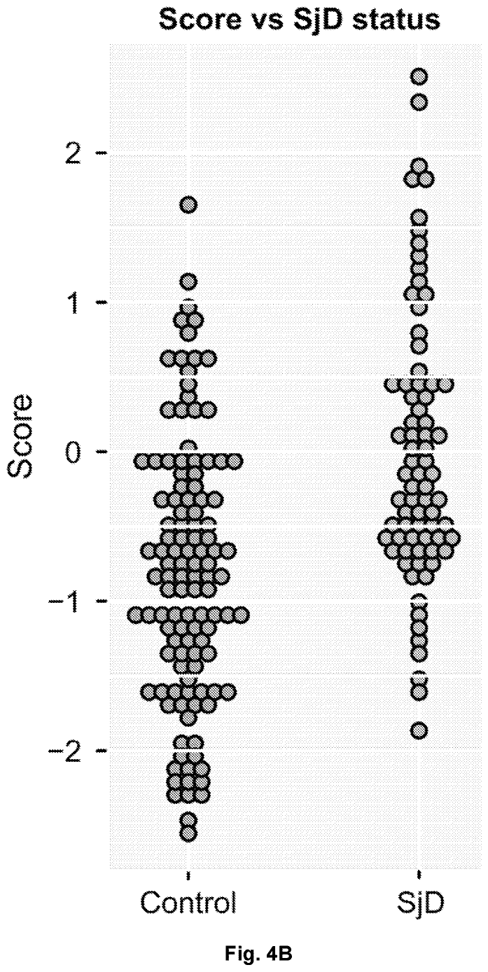

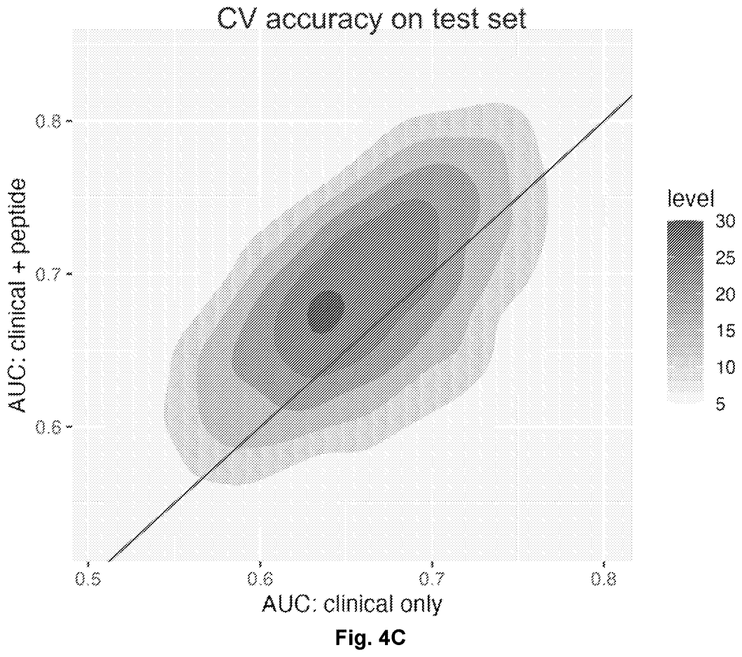

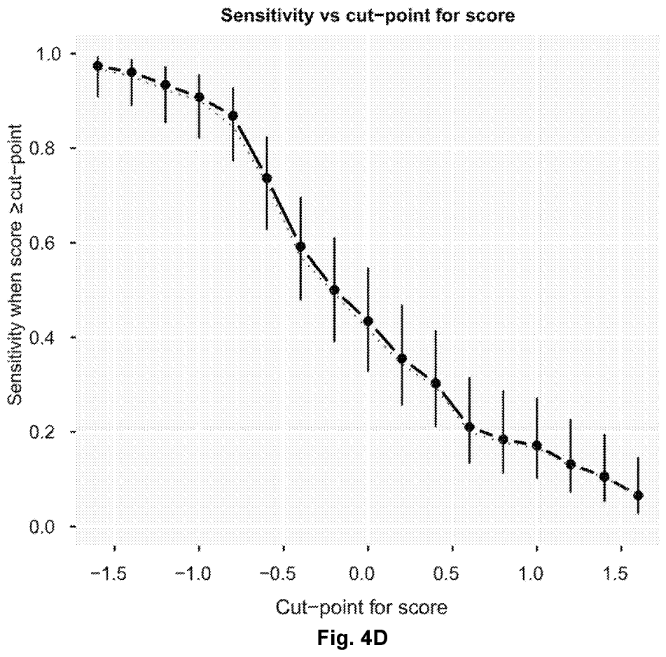

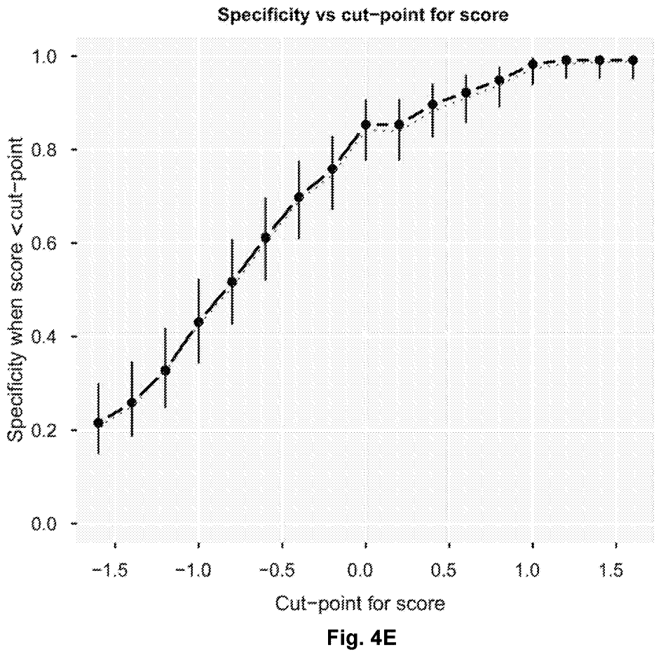

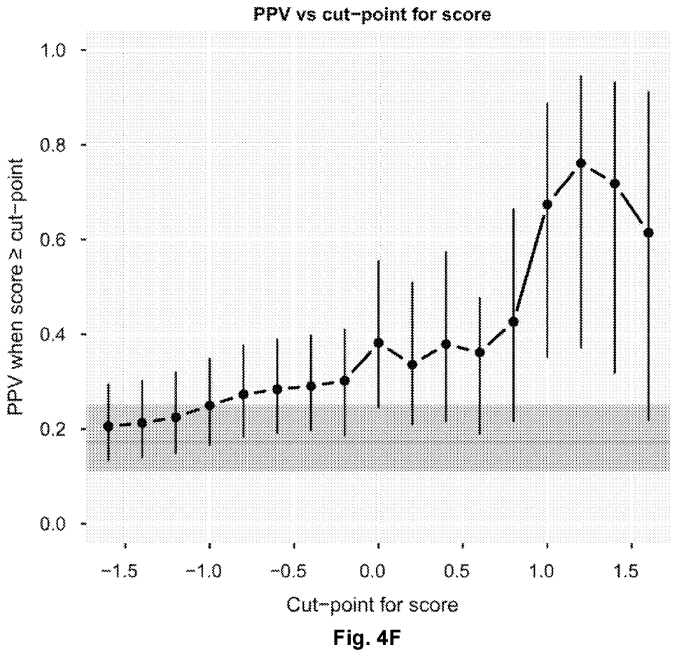

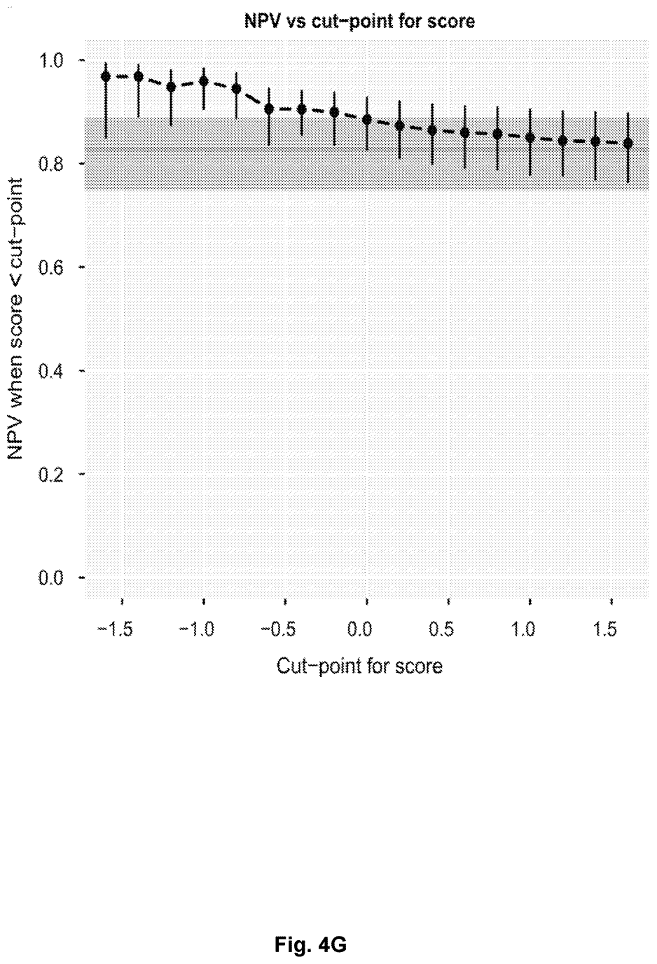

The results showed that IgG against a peptide from DTD2 (D-aminoacyl-trna deacylase 2) was greater in SSA− Sjögren's than sicca controls (p=0.0040) and a pooled control of sicca and autoimmune control patients (p=0.003) (See FIGS. 3A-30). IgG against RESF1 (Retroelement silencing factor 1) was higher in SSA− Sjögren's than sicca controls (p=0.047) and a pooled control of sicca and autoimmune control patients (p=0.03) (FIGS. 4A and 4B). We generated a regression model to predict SSA− SjD by incorporating IgG binding to our peptides into a model with clinical variables. The final model included IgG to DTD2, unstimulated salivary flow, and ANA (other peptide binding and clinical factors did not add to the model; FIG. 4C). This SjD prediction score discriminated between SSA− SjD and control subjects. Area under the ROC curve (C-index) was 73.5% (95% CI: 66.0-79.9%), which decreased to 72.2% after adjusting for optimism, discriminating well between SjD and combined controls (FIG. 4D). Sensitivity, specificity, positive predictive value, and negative predictive value are shown in FIGS. 4E-4H).

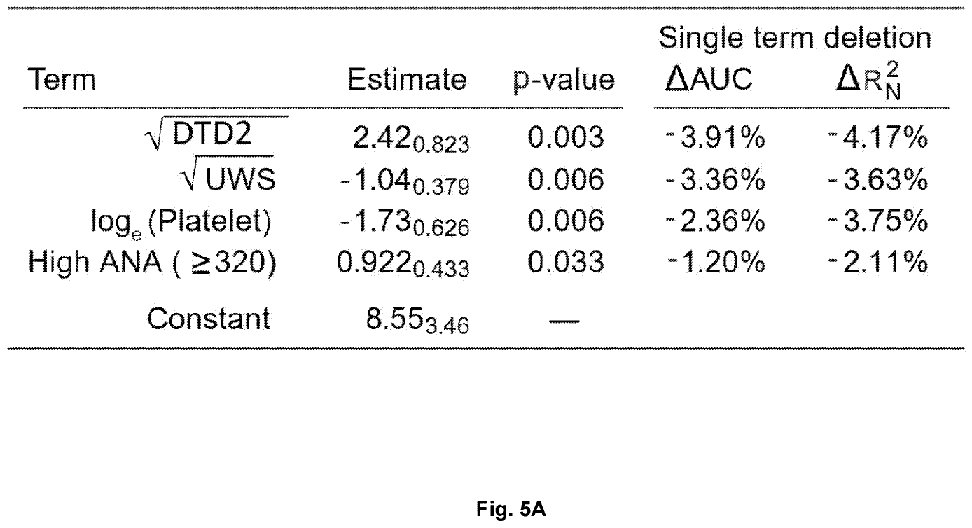

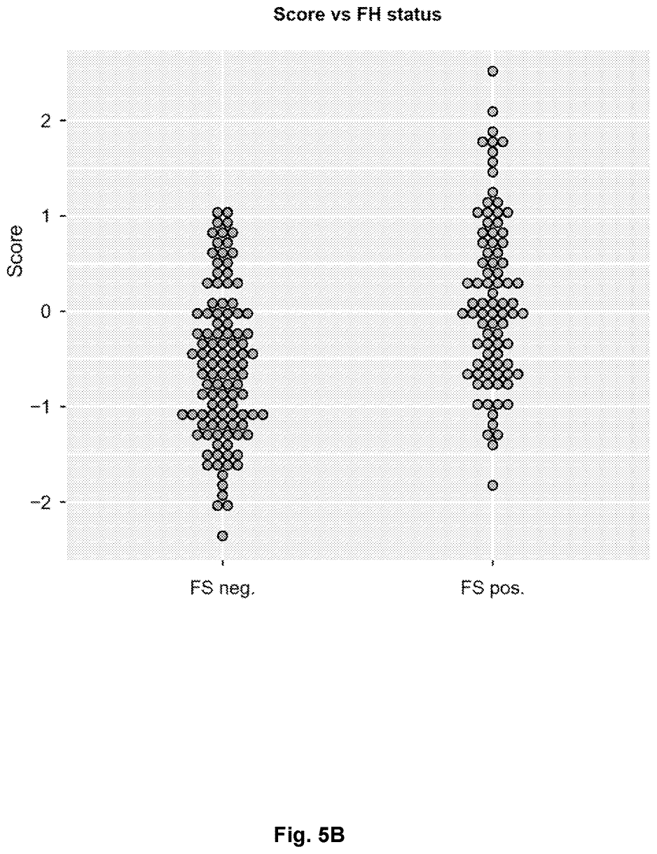

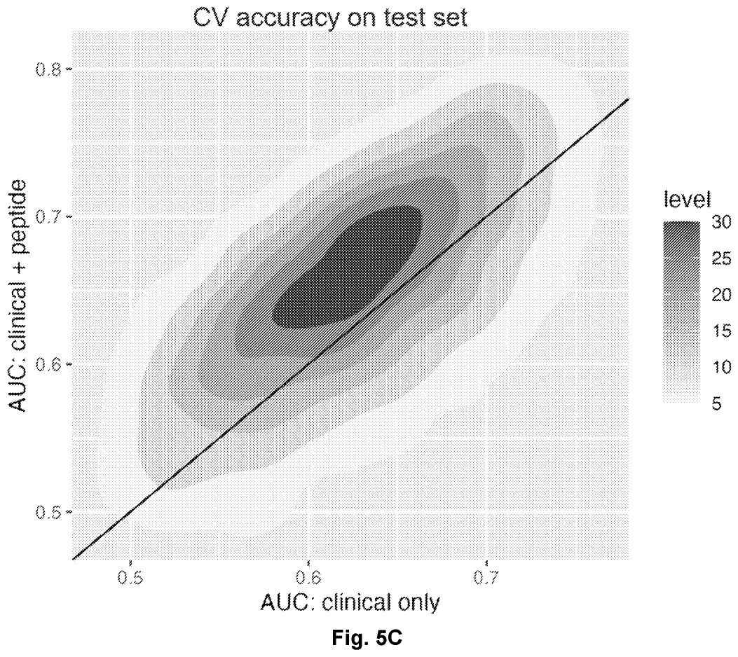

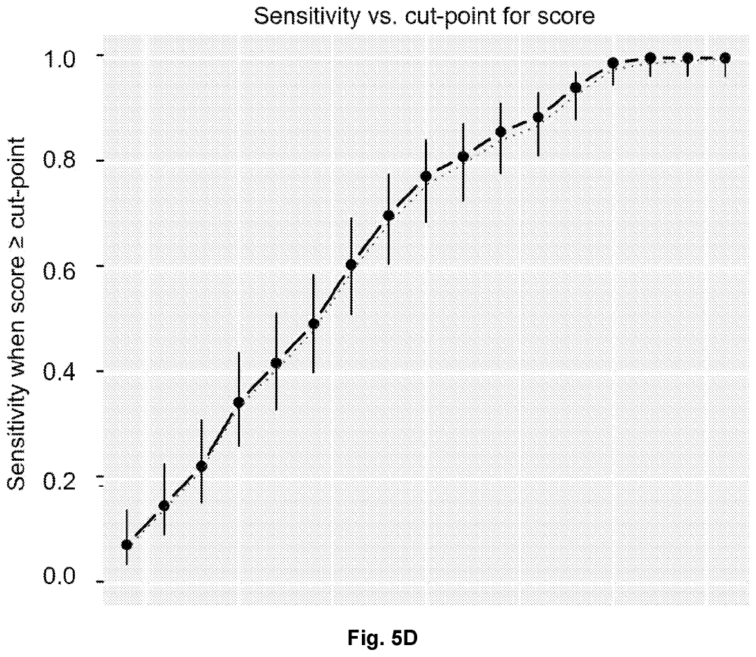

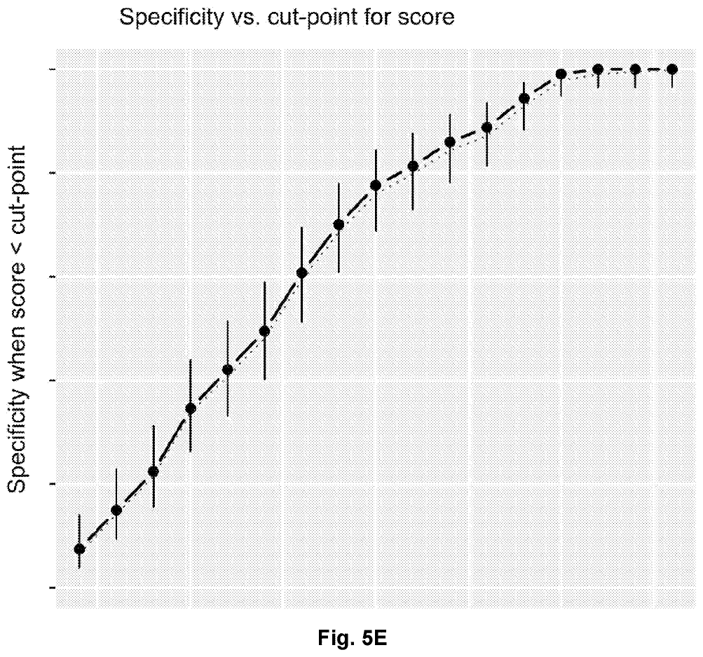

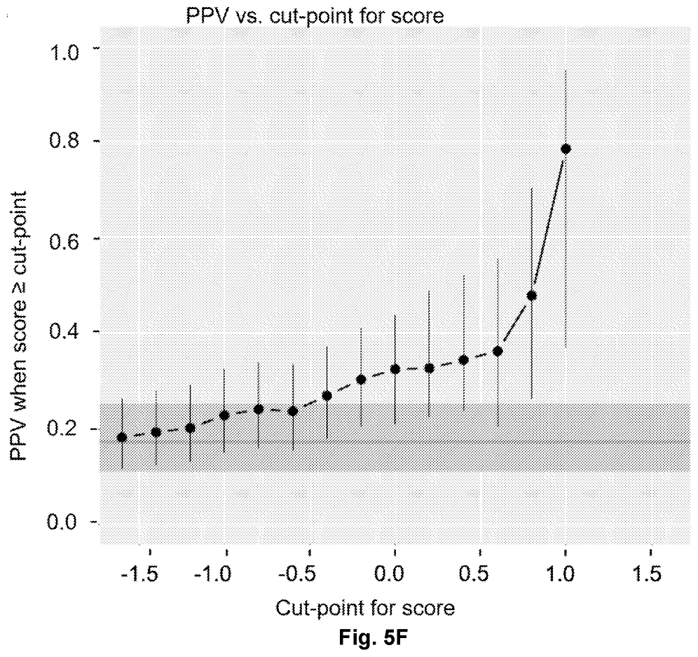

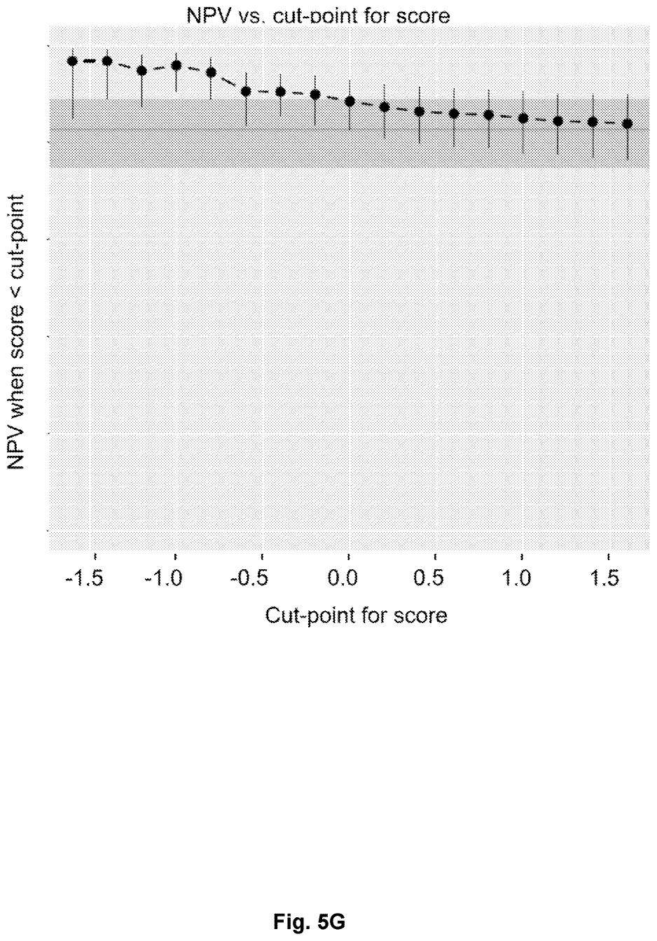

Because a surrogate marker for a positive or negative labial salivary gland biopsy is a significant clinical need, we evaluated whether autoantibody binding to the 15 peptides differed between subjects who had a positive biopsy (FS≥1) compared to a negative FS on biopsy (FS<1). We found that IgG from SSA− SjD subjects bound peptides from RESF1, DTD2, and SCRB2 more than sera from combined control subjects (p=0.01, p=0.01, p=0.03, respectively; FIG. 5A). IgG to RESF1 and DTD2 both had an estimated 61% chance that adjusted OD would be higher for a positive than a negative FS (95% CI: 53-68% and 52-68%, respectively). IgG to SCRB2 had an estimated 59% chance that adjusted OD would be higher for a positive than negative FS (95% CI: 51-67%).

We generated a regression model incorporating IgG binding to our peptides with clinical variables. The final model included IgG to DTD2, unstimulated salivary flow, platelet count, and ANA (FIG. 5B). The C-index of the model was 71.6% (95% CI: 63.9-78.2%) and decreased to 69.3% after adjusting for optimism (FIG. 5C). Binding to DTD2 contributed the most to the model (single term deletion of DTD2 yielded a more than 3.9% reduction in AUC) and the second most important was unstimulated salivary flow (single term deletion of unstimulated salivary flow yielded a more than 3.3% reduction in AUC). This final “FS prediction score” discriminated between FS positive and negative.

We calculated sensitivity and specificity for FS prediction score cut-points (range −1.6 to 1.6). Positive likelihood ratios could only be computed for cut-points ranging between −1.6 to 1.0, since none of the FS-positive group had calculated scores over 1.02 (FIGS. 5D-5G).

We present novel autoantibodies in SSA− SjD compared to autoimmune- and sicca-controls that can be used to predict disease and an abnormal FS on labial salivary gland biopsy with good predictive value.

TABLE 1
Sequences.

| | | Gene | Peptide Sequence | Peptide Sequence | Overlapping Sequence |
|---|---|---|---|---|---|
| Protein ID | Protein Name | Name | (Validated by ELISA) | (Identified by array) | (Identified by Array) |
| Q96FN9 | D-aminoacyl-trna | DTD2 | CFFKGADKELLPKMVN | YVCFFKGADKELLPKM (SEQ ID NO: 2) | CFFKGADKELLPKM |
| | deacylase 2 | | (SEQ ID NO: 1) | | (SEQ ID NO: 3) |
| Q9HCM1 | Retroelement silencing | RESF1 | REPEKQLDNTTENKDF | PEKQLDNTTENKDFGF (SEQ ID NO: 5) | LDNTTENKDF |
| | factor 1 | | (SEQ ID NO: 4) | KQLDNTTENKDFGFQK (SEQ ID NO: 6) | (SEQ ID NO: 8) |
| | | | | LDNTTENKDFGFQKDK (SEQ ID NO: 7) | |
| Q9Y6L6 | Solute carrier organic | SO1B1 | KETNINSSENSTSTLS | YRYSKETNINSSENST (SEQ ID NO: 10) | TNINSSENST |
| | anion transporter | | (SEQ ID NO: 9) | YSKETNINSSENSTST (SEQ ID NO: 11) | (SEQ ID NO: 14) |
| | family member 1B1 | | | TNINSSENSTSTLSTC (SEQ ID NO: 12) | |
| | | | | EKDINASENGSVMDEA (SEQ ID NO: 13) | |
| P22680 | Cytochrome P450 7A1 | CYP7A1 | SIDPMDGNTTENINDT | HRSIDPMDGNTTENIN (SEQ ID NO: 16) | MDGNTTENIN |
| | | | (SEQ ID NO: 15) | DPMDGNTTENINDTFI (SEQ ID NO: 17) | (SEQ ID NO;19) |
| | | | | MDGNTTENINDTFIKT (SEQ ID NO: 18) | |
| Q9C099 | Leucine-rich repeat and | LRCC1 | DDQILQLLNETSNSID | QILQLLNETSNSIDNV (SEQ ID NO: 21) | QILQLLNETSNSID |
| | coiled-coil domain- | | (SEQ ID NO: 20) | | (SEQ ID NO: 22) |
| | containing protein 1 | | | | |
| Q8NG31 | Kinetochore scaffold 1 | KNL1 | NFSEIENQTQNAMDVT | TGNFSEIENQTQNAMD (SEQ ID NO: 24) | IENQTQNAMD |
| | | | (SEQ ID NO: 23) | SEIENQTQNAMDVTTG (SEQ ID NO: 25) | (SEQ ID NO: 27) |
| | | | | IENQTQNAMDVTTGYG (SEQ ID NO: 26) | |
| Q9NS16 | Bromodomain and WD | BRWD1 | ELSNTSENDEQNAEDL | HCTNISELSNTSENDE (SEQ ID NO: 29) | ELSNTSENDE |
| | repeat-containing | | (SEQ ID NO: 28) | TNISELSNTSENDEQN (SEQ ID NO: 30) | (SEQ ID NO: 32) |
| | protein 1 | | | ISELSNTSENDEQNAE (SEQ ID NO: 31) | |
| Q9H2G2 | STE20-like | SLK | QAINSSENIMDINEEP | KNKEQAINSSENIMDI (SEQ ID NO: 34) | QAINSSENIMDI |
| | serine/threonine- | | (SEQ ID NO: 33) | KEQAINSSENIMDINE (SEQ ID NO: 35) | (SEQ ID NO: 36) |
| | protein kinase | | | | |
| Q8NEN9 | PDZ domain- | PDZD8 | SPKHTPNTSDNEGSDT | EPSPKHTPNTSDNEGS (SEQ ID NO: 38) | TPNTSDNEGS |
| | containing protein 8 | | (SEQ ID NO: 37) | KHTPNTSDNEGSDTEV (SEQ ID NO: 39) | (SEQ ID NO: 41) |
| | | | | TPNTSDNEGSDTEVCG (SEQ ID NO: 40) | |
| Q9UKV0 | Histone deacetylase 9 | HDAC9 | SLHRYDEGNFFPGSGA | | |
| | | | (SEQ ID NO: 42) | | |
| P50851 | Lipopolysaccharide- | LRBA | TLEETLTNETRNADDL | EGTLEETLTNETRNAD (SEQ ID NO: 44) | EETLTNETRNAD |
| | responsive and beige- | | (SEQ ID NO: 43) | EETLTNETRNADDLEV (SEQ ID NO: 45) | (SEQ ID NO: 49) |
| | like anchor protein | | | STKTVMDFVNSSDNVI (SEQ ID NO: 46) | VMDFVNSSDNVI |
| | | | | KTVMDFVNSSDNVIFV (SEQ ID NO: 47) | (SEQ ID NO: 50) |
| | | | | VMDFVNSSDNVIFVHN (SEQ ID NO: 48) | |
| Q14207 | Protein NPAT | NPAT | FKSEDSAVNNTQNEDG | SEDSAVNNTQNEDGIA (SEQ ID NO: 52) | DSAVNNTQNEDG |
| | | | (SEQ ID NO: 51) | DSAVNNTQNEDGIAFS (SEQ ID NO: 53) | (SEQ ID NO: 54) |
| Q9BXT5 | Testis-expressed | TEX15 | TDGNETNVTENYELDV | LHTDGNETNVTENYEL (SEQ ID NO: 56) | GNETNVTENYEL |
| | protein 15 | | (SEQ ID NO: 55) | GNETNVTENYELDVAS (SEQ ID NO: 57) | (SEQ ID NO: 58) |
| Q9HCL2 | Glycerol-3-phosphate | GPAT1 | RDTSINESRNATDESL | EGRDTSINESRNATDE (SEQ ID NO: 60) | RDTSINESRNATDE |
| | acyltransferase 1, | | (SEQ ID NO: 59) | FATNVTENVLNSSRVQ (SEQ ID NO: 61) | (SEQ ID NO: 62) |
| | mitochondrial | | | | FATNVTENVLNSSRVQ |
| | | | | | (SEQ ID NO: 63) |
| Q14108 | Lysosome membrane | SCRB2 | ILANTSDNAGFCIPEG | YKVPAEILANTSDNAG (SEQ ID NO: 65) | ILANTSDNAG |
| | protein 2 | | (SEQ ID NO: 64) | VPAEILANTSDNAGFC (SEQ ID NO: 66) | (SEQ ID NO: 67) |

TABLE 2
Demographics of the array and the SICCA registry subjects.
Peptidome Array

| | SSA- Sjögrens | Control |
|---|---|---|
| | (n = 8) | (n = 8) |
| Age mean (SD) | 58 (12) | 59 (10) |
| Female n (%) | 8 (100) | 8 (100) |
| White n (%) | 8 (100) | 8 (100) |
| Hispanic n (%) | 0 | 0 |

External Validation

| | | Autoimmune | Sicca |
|---|---|---|---|
| | SSA- Sjögrens | Control | Control |
| | (n = 76) | (n = 38) | (n = 75) |
| Age mean (SD) | 55 (12) | 55 (12) | 55 (12) |
| Female Sex n (%) | 65 (86) | 33 (87) | 64 (85) |
| White/Hispanic n (%) | 45 (59) | 24 (63) | 41 (55) |
| Other race n (%) | 31 (41) | 14 (37) | 34 (45) |

What is claimed is:

1. A method for detecting Sjögren's Disease or predicting labial salivary gland biopsy results, the method comprising:
a) providing a liquid sample obtained from an individual;
b) contacting the sample with a peptide or whole protein or fragment thereof that comprises an amino acid sequence selected from the group consisting of SEQ ID NOS: 1-67 or an epitope from the peptide, whole protein, or fragment thereof containing these sequences, under conditions appropriate to form a complex between at least a portion of any antibodies in the sample that are specific for one or more of the peptide, whole protein, or fragment thereof; and
c) correlating an amount of the complex formed in step b) to detection of Sjögren's Disease or predicting labial salivary gland biopsy results in the individual.

2. The method of claim 1, wherein the liquid sample is whole blood.

3. The method of claim 1, wherein the liquid sample is blood plasma.

4. The method of claim 1, wherein the liquid sample is blood serum.

5. The method of claim 1, wherein the sample is contacted with the protein in an enzyme-linked immunosorbent assay format.

6. A kit comprising, in combination, at least one peptide or whole protein or fragment thereof comprising an amino acid sequence selected from the group consisting of SEQ ID NOS: 1-67 adhered to a support, reagents suitable for performing an enzyme-linked immunosorbent assay, and directions for use of the kit.
